(19) United States
(12) Patent Application Publication
(10) Pub. No.: US 2021/0009767 A1
(43) Pub. Date: Jan. 14, 2021

(54) SOLVENTLESS PARTICLE COATING VIA ACOUSTIC MIXING

(71) Applicant: International Flavors & Fragrances Inc., New York, NY (US)

(72) Inventors: Richard John McCARTHY, Jr., Union Beach, NJ (US); Christopher LAVALLEE, Union Beach, NJ (US)

(21) Appl. No.: 16/925,717

(22) Filed: Jul. 10, 2020

Related U.S. Application Data
(60) Provisional application No. 62/872,918, filed on Jul. 11, 2019.

(51) Int. Cl. C08J 3/12; C08J 3/20; C08K 5/11; C08K 3/40; C08K 13/02; A61Q 13/00; A61K 8/11; A23L 27/00; A23L 29/30; A23P 20/10

(52) U.S. Cl. CPC C08J 3/126; C08J 3/203; C08K 5/11; C08K 3/40; C08K 13/02; A23V 2002/00; A61K 8/11; A23L 27/72; A23L 29/30; A23P 20/105; A23P 20/11; A61Q 13/00

(57) ABSTRACT

A method for coating solid granules containing a carbohydrate, gum Arabic, or protein by combining the solid granules with at least one solid coating material, and applying acoustic energy to said combination is provided as are coated solid granules prepared by the method.

### INTRODUCTION

This application claims the benefit of priority from U.S. Provisional Application Ser. No. 62/872,918 filed Jul. 11, 2019, the teachings of which are herein incorporated by reference in its entirety.

### BACKGROUND

Acoustic mixing is a mixing technology for liquid-liquid, liquid-solid, and powder blending. Acoustic mixing uses low-frequency sound waves (approximately 60 Hz) to induce particle or fluid motion and cause two or more components to mix into each other. In this manner, very small mixing zones (diameter of approximately 50 μm) are induced in the material, resulting in high-uniformity blends in short mixing times. Because mixing is induced through conduction of sound waves alone, no parts of the mixing unit come into direct contact with the materials being processed. This eliminates any possibility of product contamination and requires no equipment cleaning after a batch is processed.

Acoustic mixing has been applied in a number of different fields including industrial applications and coating of active pharmaceutical ingredients (API). More specifically, micronized wax and polymer particles have been combined with large ascorbic acid granules and acoustically mixed for an extended period of time (up to five hours) (Capece & Davé (2014) Powder Technol. 261:118-132; Capece, et al. (2015) J. Pharm. Sci.104(4):1340-51). Further, U.S. Pat. No. 9,107,851 discloses a solventless method of producing a polymer coated API such as ascorbic acid using high energy vibrations or acoustic mixing of API particles, soluble and/or swellable coating material particles and substantially water insoluble polymer particles. Moreover, US 2015/0290135 discloses a process for preparing free-flowing agglomerates by providing a dry powder mixture of one, two, or three APIs, and at least one excipient and applying acoustic energy to said dry powder mixture to form agglomerates.

### SUMMARY OF THE INVENTION

This invention provides a method for coating solid granules containing a carbohydrate, gum Arabic, or protein by (a) combining said solid granules with at least one solid coating material, and (b) inducing vibrations in the combination of the solid carbohydrate granules and the solid coating material via acoustic energy. In some aspects, the carbohydrate of the solid granules comprises starch, maltodextrin, sugar, polyol, cellulose, cellulose derivatives, or a combination thereof. In other aspects, the at least one solid coating material comprises gum Arabic, cellulose, a cellulose derivative, a wax, a fat, polyol, sugar, protein or a combination thereof. While the above referenced ingredients may be used alone in the preparation of coated solid granules, the method may also include the use of a plasticizer (e.g., an organic citrate salt, triglyceride, glycerol derivative, or a combination thereof) and/or impaction media (e.g., glass beads, metal balls, or a combination thereof). Moreover, one or both of the solid granules or the solid coating material may include an encapsulated flavor or encapsulated fragrance. Coated solid granules produced by the method of this invention are also provided.

### BRIEF DESCRIPTION OF THE DRAWINGS

FIG. 1 depicts dissolution results for coated carbohydrate-based extruded granule samples.

FIG. 2 depicts dissolution results for coated carbohydrate-based spray-dried powder samples.

### DETAILED DESCRIPTION OF THE INVENTION

Coating operations have conventionally been conducted using fluidized bed coating or pan coating, both of which require the coating material to be dissolved in a liquid medium and sprayed onto the solid substrate. However, the use of organic solvents complicates and increases the cost of a coating system, decreases process safety (relative to aqueous and solid coating systems) and requires costly solvent recovery and exhaust scrubbing equipment. It has now been found that the coating of flavor or fragrance solid powders or granules with a polymer or gum layer can be carried out in in a solvent-free manner using acoustic energy.

Accordingly, this invention provides a method for coating solid granules containing a carbohydrate, gum Arabic, or protein by combining solid granules containing a carbohydrate, gum Arabic, or protein with at least one solid coating material, and inducing vibrations in the combination of the solid carbohydrate granules and the solid coating material via acoustic energy thereby coating the solid granules. In accordance with this invention, the method is carried out in the absence of a solvent such as water or organic solvent, and is therefore a mechanical, dry process.

In a first step of the method of the present invention, the solid granule core is mixed with at least one solid coating material. A “solid granule” refers to a solid, dry composition composed of particles having an average particle size (diameter for substantially spherical particles) in the range of from 10 μm to 2000 μm, in a range of from 100 μm to 1500 μm, or in a range of from 200 μm to 1000 μm, as determined by sieve selection. In case of cylindrical or rod-like granules, the length preferably is about the same size ranges as described for the diameter of the particles. As used herein, the term “dry” refers material having a moisture content of no greater than 8% by weight.

The solid granules of the invention may be composed of a single ingredient or a mixture of ingredients. Further, the solid granules may constitute a carrier matrix encapsulating a flavor or fragrance therein. In certain embodiments, the carrier matrix is water soluble of the solid granule. The carrier matrix of the solid granule core may also include two or more flavor or fragrance ingredients, either in separate granules or in the same granules. This may offer advantages for combining flavors or fragrances where two flavors or fragrances may be formulated into the same formulation.

In some embodiments, combinations of solid granule cores having significantly different particle sizes may be employed. For example, two sizes of solid granule cores may be employed wherein one size of solid granule core is 1-100 times, or 3-10 times the size of the other solid granule core. For example, one size of solid granule core may have an average particle size in the range of from 750-1000 μm and the other solid granule core may have an average particle size in the range of from 250 μm to 500 μm.

In certain embodiments, the solid granule core is composed of at least one carbohydrate, at least one gum Arabic, at least one protein, or a combination thereof, wherein the solid granule core preferably encapsulates a flavor or fragrance. According to certain embodiments, the flavor or fragrance is homogeneously embedded in the carrier matrix in a manner by which the powder or amorphous solid is solubilized, and the dispersability in the matrix is uniform, so that the matrix is a monolithic entity, made up of an even homogeneous distribution of the various ingredients; including the flavor or fragrance and carbohydrate, gum Arabic, and/or protein carrier matrix.

Carbohydrates of use in the solid granule core of this invention include starch, maltodextrin, sugar, polyol, cellulose, cellulose derivatives, or a combination thereof. Exemplary carbohydrates include, but are not limited to, sucrose, glucose, dextrose, lactose, levulose, fructose, maltose, ribose, dextrose, isomalt, sorbitol, mannitol, xylitol, lactitol, maltitol, pentatol, arabinose, pentose, xylose, galactose, starch, hydrogenated starch hydrolysates, modified starch (e.g., octenylsuccinated starch), dextran, dextrin, maltodextrin, agar, carrageenan, other gums, polydextrose, a cellulose (e.g., sodium carboxymethylcellulose, hydroxypropylcellulose, methylcellulose, hydroxypropyl methylcellulose, ethylcellulose), glucose syrup solids, corn syrup solids and derivatives and mixtures thereof.

“Starch” generally refers to a mixture of linear amylose and branched amylopectin polymer of D-glucose units. The amylose is a substantially linear polymer of D-glucose units joined by (1,4)-α-D links. The amylopectin is a highly branched polymer of D-glucose units joined by (1,4)-α-D links and (1,6)-α-D links at the branch points. Naturally occurring starch typically contains relatively high levels of amylopectin, for example, corn starch (64-80% amylopectin), waxy maize (93-100% amylopectin), rice (83-84% amylopectin), potato (about 78% amylopectin), and wheat (73-83% amylopectin). As used herein, “starch” includes any naturally occurring unmodified starch, modified starch, synthetic starch or a combination thereof, as well as mixtures of the amylose or amylopectin fractions. Starch may be modified by physical, chemical, or biological processes, or combinations thereof. For example, the starch may be an octenyl succinic acid anhydride modified starch. The choice of unmodified or modified starch may depend on the end product desired. In one embodiment, the starch or starch mixture has an amylopectin content from about 20% to about 100%, more typically from about 40% to about 90%, even more typically from about 60% to about 85% by weight of the starch or mixtures thereof. Suitable naturally occurring starches can include, but are not limited to, corn starch, potato starch, sweet potato starch, wheat starch, sago palm starch, tapioca starch, rice starch, soybean starch, arrow root starch, amioca starch, bracken starch, lotus starch, waxy maize starch, and high amylose corn starch.

“Dextrin” is a water-soluble polysaccharide obtained from starch by the action of heat, acids, or enzymes. The term “dextrin,” in its broadest sense, may refer to any product obtained by any method (e.g., heat, acid, enzyme) for degrading the starch. The tensile strength of dextrin film is lower than that for starch and decreases with the degree of conversion.

“Dextran” is a complex branched polysaccharide synthetized from sucrose by certain lactic-acid bacteria, e.g., Leuconostoc bacteroides and Streptococcus mutans. Dextran chains are of varying lengths (from 3 to 2000 KDa) and are composed of α-1,6 glycosidic linkages between glucose monomers, with branches from α-1,3 linkages. This characteristic branching distinguishes a dextran from a dextrin, which is a straight chain glucose polymer tethered by α-1,4 or α-1,6 linkages.

“Cellulose” is a complex carbohydrate or polysaccharide, composed of a linear chain of β-1,4 linked D-glucose units. Cellulose is the main substance found in plant cell walls, but is also produced by some bacteria. However, unlike plant-based cellulose, bacterial cellulose is highly pure and does not need to be separated from lignin in processing. Accordingly, in some embodiments, the cellulose used in the preparation of the solid granule core of this invention is a plant cellulose, whereas in other embodiments, the cellulose used in the preparation of the solid granule core is a bacterial cellulose.

As is known in the art, modification of cellulose by etherification chemistries increases the water solubility of cellulose by decreasing the crystallinity of the cellulose molecule. Accordingly, in certain embodiments of this invention, the cellulose is a modified cellulose, in particular a cellulose ether. Examples of modified celluloses include, but are not limited to, carboxymethylcellulose, hydroxyethylcellulose, carboxymethyl hydroxyethyl cellulose, hydroxypropyl cellulose, ethyl hydroxyethyl cellulose, methyl ethyl hydroxyethyl cellulose, or salts or a combination thereof.

The term “maltodextrin” refers to a particular group of carbohydrates such as different lengths/complexity starch degradation products, rather than a single compound having a specific chemical structure. U.S. Food and Drug Administration describes maltodextrin as being composed of mainly [α]-1,4 D-glucose units and having 20 or fewer dextrose equivalent (DE). Maltodextrin can be prepared from a variety of materials in various ways. Preferred for use herein are maltodextrins having about 4 to 20 DE, which is derived from starch such as corn starch, potato starch, rice starch and the like.

According to a preferred embodiment of the invention, there will be used maltodextrin or mixtures of maltodextrin and at least one material selected from the group of sucrose, glucose, dextrose, lactose, levulose, maltose, fructose, isomalt, sorbitol, mannitol, xylitol, lactitol, maltitol and modified starch. In other embodiments, mixtures of different types of maltodextrins are used, e.g., a combination of a DE 10 maltodextrin and a DE 15 maltodextrin. Preferably a maltodextrin having a dextrose equivalent not above twenty (≤20 DE) and more preferably a DE in the range of 10 to 15 is used.

In further embodiments, corn syrup solids are used. Corn syrup solids refers to a group of carbohydrates with DE greater than 20. Of particular use in this invention are corn syrup solids having a DE in the range of 21 to 48.

In some embodiments, the solid granule core includes at least one polypeptide or protein. In other embodiments, the solid granule core includes at least two, three, four, five or more proteins. In this respect, a protein of use in the solid granule core of the invention can be a single, individual protein or a combination of proteins. Exemplary protein and protein combinations include, but are not limited to; gelatin, whey protein (e.g., a concentrate or isolate), plant storage protein (e.g., a concentrate or isolate), or a combination thereof.

As used herein, “whey protein” refers to the proteins contained in whey, a dairy liquid obtained as a supernatant of curds when milk or a dairy liquid containing milk components, is processed into cheese curd to obtain a cheese-making curd as a semisolid. Whey protein is generally understood in principle to include the globular proteins β-lactoglobulin and a-lactalbumin. It may also include lower amounts of immunoglobulin and other globulins. The term “whey protein” is also intended to include partially or completely modified or denatured whey proteins. Purified β-lactoglobulin and/or α-lactalbumin proteins may also be used in preparation of the solid granule core of this invention.

Plant storage proteins are proteins that accumulate in various plant tissues and function as biological reserves of metal ions and amino acids. Plant storage proteins can be classified into two classes: seed or grain storage proteins and vegetative storage proteins. Seed/grain storage proteins are a set of proteins that accumulate to high levels in seeds/grains during the late stages of seed/grain development, whereas vegetative storage proteins are proteins that accumulate in vegetative tissues such as leaves, stems and, depending on plant species, tubers. During germination, seed/grain storage proteins are degraded and the resulting amino acids are used by the developing seedlings as a nutritional source. In some embodiments, the plant storage protein used in the preparation of a solid granule core of the invention is a seed or grain storage protein, vegetable storage protein, or a combination thereof. In certain embodiments, the seed storage protein is a leguminous storage protein. In particular embodiments, the seed/grain storage protein is extracted from leguminous plants and particularly from soya, lupine, pea, chickpea, alfalfa, horse bean, lentil, and haricot bean; from oilseed plants such as colza, cottonseed and sunflower; from cereals like wheat, maize, barley, malt, oats, rye and rice (e.g., brown rice protein), or a combination thereof. In other embodiments, the plant storage protein is a vegetable protein extracted from potato or sweet potato tubers.

In particular embodiments, the plant storage protein is intended to include a plant protein isolate, plant protein concentrate, or a combination thereof. Plant storage protein isolates and concentrates are generally understood to be composed of several proteins. For example, pea protein isolates and concentrates may include legumin, vicilin and convicilin proteins. Similarly, brown rice protein isolates may include albumin, globulin and glutelin proteins. The term “plant storage protein” is also intended to include a partially or completely modified or denatured plant storage protein. Individual storage polypeptides (e.g., legumin, vicilin, convicilin, albumin, globulin or glutelin) may also be used in preparation of the solid granule core of this invention.

“Gelatin” refers to a mixture of proteins produced by partial hydrolysis of collagen extracted from the skin, bones, and connective tissues of animals. Gelatin can be derived from any type of collagen, such as collagen type I, II, III, or IV. Such proteins are characterized by including Gly-Xaa-Yaa triplets wherein Gly is the amino acid glycine and Xaa and Yaa can be the same or different and can be any known amino acid. At least 40% of the amino acids are preferably present in the form of consecutive Gly-Xaa-Yaa triplets.

The protein used in the solid granule core can also be derivatized or modified (e.g., derivatized or chemically modified). For example, the protein can be modified by covalently attaching sugars, lipids, cofactors, peptides, or other chemical groups including phosphate, acetate, methyl, and other natural or unnatural molecule.

In some embodiments, the solid granule core includes gum Arabic. Gum Arabic is a complex mixture of arabinogalactan oligosaccharides, polysaccharides, and glyco-proteins. It is a branched neutral or slightly acidic substance. The chemical composition and the composition of the mixture can vary with the source, climate, season, age of trees, rainfall, time of exudation, and other factors. The backbone has been identified to be composed of β-(1→3)-linked D-galactopyranosyl units. The side chains are composed of two to five p-(1→3)-linked D-galactopyranosyl units, joined to the main chain by 1,6-linkages. Both the main and the side chain contain units of α-L-arabinofuranosyl, α-L-rhamnopyranosyl, β-D-glucuronopyranosyl, and 4-O-methyl-β-D-glucuronopyranosyl, the latter two of which usually occur preferably as end-units. Depending on the source, the glycan components of gum Arabic contain a greater proportion of L-arabinose relative to D-galactose (Acacia seyal) or D-galactose relative to L-arabinose (Acacia senegal). The gum from Acacia seyal also contains significantly more 4-O-methyl-D-glucuronic acid but less L-rhamnose and unsubstituted D-glucuronic acid than that from Acacia senegal.

As with the solid granule, the solid coating material refers to a solid, dry composition. The solid coating material may be in the form of a particle, powder or micronized material having a volume-weighted mean particle size in the range of from 100 nm to 1000 μm or more preferably in the range of from 1 μm to 500 μm.

For delayed-release of the solid granule core, the solid coating material is ideally slowly soluble in water or insoluble in water, and may optionally be capable of being degraded within the GI tract by means including enzymatic degradation, surfactant action of bile acids, and mechanical erosion or alternatively provides release based upon a change in pH (e.g., the solid coating material protects the core at higher pH but releases the core material under the acidic conditions of the stomach). As used herein, the term “slowly soluble in water” refers to materials that are not dissolved in water within a period of 1 to 5 minutes. Alternatively stated, the coatings permit at least 90% of the release of the uncoated solid granule core at 5 minutes in a standard dissolution test.

Solid coating materials of use in this invention include gum Arabic, cellulose, a cellulose derivative, polyol, sugar, or protein as described herein, or a wax, fat or fatty substance, or a combination of any of the above. Preferred examples of solid coating materials include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trademark STEROTEX®, stearic acid, cocoa butter, glyceryl behenate (available under the trademark COMPRITOL 888®), glyceryl dipalmitostearate (available under the trademark PRECIROL®), and stearyl alcohol. Mixtures of mono-, di- and tri-glycerides and mono- and di-fatty acid esters of polyethylene glycol, available under the trademark GELUCIRE®) are also suitable fatty materials. Suitable waxes and wax-like materials include natural or synthetic waxes, hydrocarbons, and normal waxes. Specific examples of waxes include beeswax, glycowax, castor wax, carnauba wax, polyethylene wax, paraffins and candelilla wax. As used herein, a wax-like material is defined as any material which is normally solid at room temperature and has a melting point of from about 30° C. to 300° C.

Ideally, the solid coating material is deformable under mechanical stress and optionally elevated temperature and thus is selected to have a Young's modulus of not greater than 420 MPa, or not greater than 200 MPa, or not greater than 100 MPa, as measured at 20° C. Alternatively, the deformability should be equivalent to a Young's modulus of not greater than 420 MPa or not greater than 200 MPa, or not greater than 100 MPa, as measured at 20° C. when measured at elevated or reduced temperatures actually used for processing. Thus, it is contemplated, for example, that elevated processing temperatures could be employed to soften the solid coating material for deformation or that a combination of softening at elevated temperature and mechanical stress can be employed.

The amount of solid granule core employed in the mixing step is in a range of from 10 wt. % to 90 wt. %, or from 20 wt. % to 80 wt. % of the total weight of the solid granule core and coating materials. Similarly, the amount of solid coating material employed in the mixing step is in a range of from 10 wt. % to 50 wt. %, or from 20 wt. % to 40 wt. % of the total weight of the solid granule core and coating materials.

In some cases, it may be desirable to alter the rate of water penetration into the solid granule core. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethylcellulose, hydroxypropylcellulose, methylcellulose, and carboxymethylcellulose), alginic acid, lactose and talc.

In addition, in certain embodiments, it may be beneficial to include a plasticizer to provide softening of a hard coating material, thereby enhancing the overall deformability of the coating and contributing to a greater surface coverage on the substrate particle. Examples of suitable plasticizers include organic citrate salts, triglycerides and glycerol derivatives. Particular plasticizers include, but are not limited to, triacetin, acetylated monoglyceride, rape oil, olive oil, sesame oil, acetyltributyl citrate, glycerin sorbitol, diethyloxalate, diethyl malate, diethyl fumarate, dibutyl succinate, diethylmalonate, dioctylphthalate, dibutyl succinate, triethyl citrate, tributyl citrate, glycerol tributyrate, propylene glycol, polyethylene glycols, hydrogenated castor oil, fatty acids, substituted triglycerides and glycerides, and the like.

Furthermore, the coating material may encapsulate a flavor or fragrance therein. In this respect, when the solid granule core and solid coating material each encapsulate a flavor or fragrance, said flavor or fragrance in each of the solid granule core and solid coating material may be the same or different. This may offer advantages for combining flavors or fragrances where two or more flavors or fragrances may be formulated into the same formulation and/or where controlled release profiles are desired.

Generally speaking, a surface coverage of the solid granule core with the coating material of at least 90-100% should be achieved. The theoretical surface coverage of the solid granule core may be calculated based on the particle sizes, assuming that the particles are spherical and uniform in size. To achieve a theoretical surface coverage of 100% of the solid granule core, the amount of coating material needed may be calculated. A skilled person may adjust the calculation when the particles have different shapes or are non-uniform.

In embodiments where a combination of coating materials is used, the coating materials are pre-blended to ensure more uniform contact between the solid granule core and the coating materials. Preblending is employed to produce a thorough mixture of the coating materials generally without attrition of the particles. For example, preblending may be achieved using a LabRAM acoustic mixer at 100 G's for 1 minute, or a rolling drum rotated at 60 RPM for 1 hour.

The solid granule core is then mixed with the pre-blended coating materials or the coating materials can be added batchwise or stepwise to the solid granule core. Mechanical stress is then applied to the mixture by, for example, inducing vibrations via acoustic mixing. The mixing of the ingredients in this step is sufficient disperse and discretely coat the coating material onto the solid granule core and to subsequently deform the discrete coating on the solid granule core. The collisions facilitate attachment of unattached coating material to the surface of solid granule core. Continued collisions deform the coating material, which will form a substantially continuous coating on the surface of solid granule core.

The step of inducing vibrations may be carried out for a period of from about 30 minutes to about 5 hours, depending on the characteristics of the coating materials, the size of the core particles and the loading. In some cases, especially involving fine core particles, a period of up to 4 hours may be used. A skilled person may determine an appropriate length for the mixing step by monitoring the size of the dry coated granules using images of samples taken at various times during the process. In some embodiments, the coating material may be added in a step-wise fashion to the solid granule core while the ingredients are being mixed. Step-wise addition of coating materials makes it possible to deform each coating layer individually rather than deforming just the outer layer.

The induction of vibrations is suitably carried out by acoustic mixing using low frequency, high-intensity acoustic energy transferred to the mixing chamber by propagation of acoustic pressure waves into the mixing chamber. Low frequency acoustic mixing at up 100 g of acceleration is suitable with a frequency of about 60 Hz. Acoustic mixing has the advantage of no bulk flow and mixing occurs on a micro scale throughout the mixing volume. In a typical acoustic mixing device, an oscillating mechanical driver creates motion in a mechanical system composed of engineered plates, eccentric weights and springs. This energy is then acoustically transferred to the ingredients of the mixing step in the mixing chamber. The system may be operated at resonance.

Exemplary devices for mixing include a Resodyne acoustic mixer such as LabRAM I, LabRAM II, OmniRAM, RAM 5 or RAM55 or a Design Integrated Technology, Inc. acoustic mixer such as the Sonic Mixer 2L or 20L. For example, in a LabRAM, the mixing step provides a highly efficient way of transferring mechanical energy through acoustic pressure waves directly to the ingredients in the mixing step. The resonance is achieved by matching the operational parameters of the mixer with the properties and characteristics of the materials to be mixed. In general, any device that allows sufficient number of collisions with the appropriate intensity so that polymer deformation can take place without significant attrition of the solid granule core to be coated, may be used.

The device, operating conditions, processing time, and the polydispersity of the solid granule core to be coated can be selected by those skilled in the art such that; (a) the coating is uniform and deformed sufficiently, and (b) the process does not lead to significant attrition of the solid granule core. In a selected process, the coating material should be dispersed over the solid granule core surface and subsequently mechanically deformed by, for example, vibrating and optional impaction in a suitable mixer providing sufficient stress due to impactions in order to result in polymer deformation. Stress may result from mechanical interactions of the particles themselves, impaction media, the vessel wall and/or other parts of the mixer. Suitable equipment may cause impactions by particles, media, or vessel geometry with relative velocities of about 0.01-10 m/s, or about 1-5 m/s. Such velocities of impact may not be easily measured, but can be estimated by computer simulation.

Alternately, the effect of processing can be quantified via the performance of the coated product in various other manners. The mixing intensity should be high enough so that the coating material will deform, but is not excessive so as to break or attrite the substrate or the coating layer that is already deformed and well-spread. Attrition can be determined by the presence of, or increase of fines as measured in a particle size analyzer or identified by an increase in release of the coated solid granule core at prolonged processing times as compared to an optimum processing time. More specifically, the average size of the coated solid granule core is expected to be about the same as or larger than the original uncoated solid granule core.

In some embodiments, the method may also include the use of impaction media to increase the number of collisions or the intensity of the collisions. The impaction media may be selected from the group of inorganic particles, glass beads, ceramic beads, metal balls such as stainless steel balls, salts, sugars, agate, and combinations thereof. In general, any material with density equal to or higher than the solid granule core density may be used as media. The sizes and types of medial are typically selected to avoid excessive attrition and to sufficiently deform the coating material. Any particle with a density of at least about 1.6 g/ml may be employed.

Ideally, the presence of impaction media improves the formation of a substantially continuous polymer coating on the solid granule core. The impaction media preferably have a significantly different median particle size than the solid granule core, with a typical ratio of median particle sizes being from 3:1 to 25:1, preferably from 3:1 to 10:1. The impaction media or the solid granule core may be selected to be the larger of the particles in different embodiments of the invention. The ratio between the number of solid granule cores and the number of impaction media in the method of the invention may be in a range of from 1:1 to 10:1. After the mixing of the ingredients, the impaction media may be separated from the coated solid granule core by sieving based on their difference in particle size. Use of different sizes of impaction media and solid granule core makes the separation efficient and simple.

Certain types of coated solid granule cores may suffer from adhesion and caking during storage. To prevent this, the present invention may include a further step of dry coating the coated solid granule core with silica. In one embodiment, the coated solid granule core is mixed with a sufficient amount of silica particles to provide at least 100% surface coverage, e.g., 1% by weight of silica particles such as Aerosil R972 fumed silica to improve the flowability of the coated solid granule core. An amount of silica in a range of from 0.1 to 2 wt %; or an amount sufficient to provide a surface area coverage from about 20 to about 100% may be used. The silica coating may be applied using a LabRAM at 50G′s for 30 seconds or by simply blending the coated solid granule core with the silica particles.

Desirably, the moisture content of the coated solid granule core is less than 10% or more preferably less than 5%. Preferably, the water activity of the coated solid granule core is less than 0.5 or more preferably less than 0.3.

The coated solid granules of this invention find use in many applications including the preparation of foods, food additives, and consumer products such as cosmetics, cleaning agents, personal care products and the like.

The following non-limiting examples are provided to further illustrate the present invention.

### EXAMPLE 1

### Materials and Methods

A LabRAM acoustic mixer (Resodyn Acoustic Mixers, Butte, Mont.) with a maximum capacity of 16 fl oz of mixing volume was used in this study. Carbohydrate-based extruded granule and spray-dried powder samples were used in order to provide clear solutions during dissolution testing. Additionally, both samples contained 0.075% w/w FD&C Blue #1 dye in order to track product dissolution in aqueous media. The carbohydrate-based extruded granule sample was milled and sifted to standard size (−18/+60 mesh, approximately 250 μm to 1000 μm) with no additional post-processing. Carbohydrate-based extruded granule and spray-dried powder were selected as model cores for this study as they have significantly different particle size distributions; in which carbohydrate-based extruded granule is regular in shape, having a rod-like appearance and a smooth surface morphology, while carbohydrate-based spray-dried powder is much smaller and more irregular in shape, having an appearance much more like a raspberry or a cluster of grapes.

Five different coating materials were used in this study with a range of average particle sizes and mechanical properties. Specifically, the coating materials included carnauba wax (Microcare 350GMP; Micro Powders Inc.), polyethylene wax (Microcare 110XXF; Micro Powders Inc.), ethylcellulose (Ethocel™ HP; Colorcon), an ethylcellulose system sold under the trademark OPADRY® EC (Colorcon) and hydrogenated cottonseed oil (07 Stearine™; IOI Loders Croklaan). All five coating systems are water-insoluble, and thus were solely responsible for delayed release of processed products. The carnauba wax (CW), polyethylene wax (PE) and ethylcellulose (EC) used were micronized powders, having volume-weighted mean particle size values (d4;3) between 6.7 μm and 8.0 μm. The ethylcellulose system (ES), which contains a mixture of ethylcellulose, talc, hypromellose and triethyl citrate, is composed of very large particles, with a mean diameter 40-50 times larger than that of the micronized powders. Additionally, the hydrogenated cottonseed oil (CO) is sold as large, flat flakes, sometimes a few millimeters in diameter; these flakes were broken up by hand to diameters of 2 mm to 3 mm before being combined with the substrates.

For the samples to be processed, each of the components (substrate, coating material, impaction media and plasticizer) was weighed into a standard 16 oz glass jar and mixed by hand using a plastic spoon for a few seconds. This premix step was done in order to prevent all of the coating material from immediately adhering to the bottom of the jar in the initial stages of acoustic mixing. The jar was then capped with a black plastic cap and clamped into the stand of the LabRAM acoustic mixer. The LabRAM was then set for a starting mixing intensity value and mixing time using the controls on the integrated user control panel. The mixer was then started using the same on-board controls and the mixing intensity adjusted to match the desired mixing acceleration for that particular sample. During the run, the intensity was manually adjusted in order to maintain a constant mixing acceleration. The mixer was then allowed to run until the desired time had elapsed, at which time the mixer automatically stopped.

Table 1 lists all of the experiments conducted using the LabRAM acoustic mixer.

TABLE 1

| | | | | | |
|---|---|---|---|---|---|
| Sample | Coating | Coating | Impaction | | Batch |
| Name | Material¹ | Level² | Media³ | Plasticizer⁴ | Size |

Carbohydrate-based extruded granule

| | | | | | |
|---|---|---|---|---|---|
| F01 | PE | 20% | None | None | 50 g |
| F12 | PE | 40% | None | None | 50 g |
| F02 | CW | 20% | None | None | 50 g |
| F11 | CW | 40% | None | None | 50 g |
| F20 | CW | 40% | None | None | 50 g |
| F03 | EC | 20% | None | None | 50 g |
| F13 | EC | 40% | None | None | 50 g |
| F14 | EC | 40% | None | 10% | 50 g |
| F04 | ES | 20% | None | None | 50 g |
| F17 | CO | 40% | None | None | 50 g |
| F18 | CO | 40% | None | None | 100 g |

Carbohydrate-based spray-dried powder

| | | | | | |
|---|---|---|---|---|---|
| F05 | PE | 30% | None | None | 50 g |
| F10 | PE | 30% | 50% | None | 50 g |
| F06 | CW | 30% | None | None | 50 g |
| F09 | CW | 30% | 50% | None | 50 g |
| F07 | EC | 30% | None | None | 50 g |
| F08 | EC | 30% | 50% | None | 50 g |
| F15 | EC | 50% | 50% | 10% | 50 g |
| F16 | EC | 50% | 50% | None | 50 g |
| F19 | CO | 50% | 50% | None | 100 g |

¹PE = polyethylene wax; CW = carnauba wax; EC = ethylcellulose; ES = ethylcellulose system; CO = hydrogenated cottonseed oil
²Expressed as % w/w of final product
³30 mesh glass beads used as impaction media; level expressed as % w/w of substrate mass
⁴Triethyl citrate used as plasticizer

All batches were run at a mixing acceleration of 100 g for four hours. The selected mixing time of four hours allowed for maximum productivity, as two samples could be processed in a standard workday. In addition to the substrate and coating material, 30 mesh glass beads were added to some samples as impaction media in an attempt to assist the deformation of the coating material during processing and enhance deposition onto the substrate surface. For certain samples using ethylcellulose as the coating material, a small amount of triethyl citrate was added to the product as a plasticizer to soften the polymer and facilitate mechanical deformation. Once the run was completed, the product jar was removed from the mixer's stand using the clamping mechanism. The jar was then opened, visually inspected and photographed, and the product temperature immediately measured and recorded. Once fully cooled down to ambient temperature, samples containing any impaction media were then filtered through a mesh screen, where applicable, and the filtrate collected as the final product.

Particle size distributions for each product were then measured using laser diffraction analysis (Mastersizer 3000, Malvern Instruments Ltd, Malvern, UK). Additionally, products were analyzed using optical microscopy (Model M10T-BTW1-MP compound microscope, Swift Optical Instruments, Inc., Schertz, Tex.) to assess particle composition, morphology and surface characteristics. Finally, product dissolution characteristics were tested by dissolving 8 g substrate equivalent (i.e., not including coating mass) in 800 g of a 0.25% w/w aqueous polysorbate 20 sold under the trademark TWEEN® 20 and sampling at set elapsed times. Samples were then allowed to settle for a minimum of 24 hours before testing; once fully settled, absorbance of each solution at 630 nm was measured using spectrophotometry (Agilent Cary 8454 UV-Vis spectrophotometer, Agilent Technologies, Santa Clara, Calif.). These three analytical methods were conducted in order to determine final product characteristics, with particular emphasis on coating integrity, surface morphology and particle agglomeration and attrition. Each of these methods gave a unique insight into the process of acoustic mixing through the properties of the product.

Particle size analysis of each sample was conducted, which compared (i) the particle size distribution of the core material (i.e., substrate), (ii) the particle size distribution of the coating material, (iii) the particle size distribution of a hand-mixed (i.e., no acoustic processing) combination of the core material and the coating material in the proper proportion, and (iv) the particle size distribution of the acoustic-processed prototype. The four curves were overlaid to show the success or failure of the acoustic coating process for that prototype, as well as allow for the identification of residual peaks in the particle size distribution of the final processed prototype. Deviations in the particle size of the product from that of the untreated substrate give an indication, when taken together with optical images and dissolution testing results, of particle agglomeration, attrition or coating. Further, changes in shape of the particle size distribution, and specifically the appearance of secondary or tertiary peaks, would indicate the presence of residual coating material in the product. Slight increases in particle size would be indicative of coating, but very large increases would indicate particle agglomeration. Narrow peaks in the range of the average diameter of the coating material would indicate the presence of residual coating material, while a broader peak in any range may be more likely indicative of particle attrition during processing.

Optical imaging of samples was also conducted to assess the surface morphology of the product particles, as well as to visually inspect for the presence of residual unadhered coating material. These microscopy images were used to detect differences in surface characteristics, including structure of the coating layer (i.e., unadhered, discrete or continuous) and coating uniformity. This method was also used to supplement particle size analysis in assessing the extent of particle agglomeration and attrition, as evidenced by particles adhered together or particles which have one or more facets with different coating structures. It was determined that more sophisticated microscopy techniques, such as scanning electron microscopy (SEM), were not required due to the size of the particles under consideration and the breadth of information obtained from the optical method alone.

In addition to particle size analysis and optical microscopy, dissolution testing was conducted on each sample to assess its controlled release properties. Results of this testing provided the most definitive information regarding the structure and porosity of the resulting coating. Because the substrates used were highly water-soluble and the coating materials highly water-insoluble, all dissolution testing was conducted at room temperature (approximately 22° C.) in a solution containing 0.25% w/w polysorbate 20 (sold under the trademark TWEEN® 20) in deionized water. This enabled assessment of the integrity of the coating by way of water diffusion into and out of the coating layer.

In order to accurately assess the results of dissolution testing, a protocol was developed combining a spectrophotometric method with statistical modeling and curve fitting. Specifically, samples of the testing solution were gathered at various time points and the absorbance of each measured at 630 nm, that being the maximum absorbance value for Blue #1 dye. Each solution's absorbance at 630 nm was then reduced by its absorbance at 800 nm in order to reduce deviations in spectral baseline heights caused by slight solution turbidity differences; this “adjusted” absorbance value was then representative of the prominence of the blue dye absorbance peak over the baseline. The time-absorbance data was then fitted to an exponential decay model of the form:

Aₘ=a−bw^{-ct} (1)

where Aₘ is the adjusted absorbance value, t is the elapsed dissolution time (s), and a, b and c are regression coefficients. Values for the regression coefficients were obtained using the minpack.lm package with the programming language, which provides robust nonlinear regression modeling methods to the base software package. Once these values were accurately calculated, each absorbance value was divided by the model's asymptote value a in order to normalize for any differences in dye concentration between prototype samples. A new model was fitted to the resulting data with only a single regression parameter:

d=1−e^{-λt} (2)

where d=Aₘ/a represents the fraction of substrate dissolved (0≤d≤1), and A is the release rate (s⁻¹). Controlled release characteristics of each prototype could then be assessed relative to one another by comparison of the release rates. To facilitate this comparison, a delay rate δ=−lnλ was often cited instead of the release rate, wherein a larger delay rate indicates a more significant delayed release of the coated prototype.

### EXAMPLE 2

### Coating of Carbohydrate-Based Extruded Granules

Particle size analysis resulting from acoustic mixing of granules as a substrate in combination with the coating materials and conditions provided in Table 1 are summarized in Table 2.

TABLE 2

| | | Dominant Mode | | Secondary Mode | |
|---|---|---|---|---|---|
| Sample | d_{4,3}, μm | Size, μm | Peak % | Size, μm | Peak % |
| Substrate | 570 | 503 | 97.17 | 66.8 | 2.22 |
| only | | | | | |
| F01 | 642 | 580 | 100.00 | — | — |
| F12 | 665 | 620 | 99.31 | — | — |
| F02 | 654 | 582 | 100.00 | — | — |
| F11 | 705 | 665 | 99.00 | — | — |
| F20 | 707 | 639 | 97.89 | — | — |
| F03 | 609 | 546 | 97.44 | 5.08 | 1.77 |
| F13 | 820 | 726 | 81.18 | 6.93 | 18.51 |
| F14 | 845 | 658 | 85.44 | 6.82 | 14.56 |
| F04 | 513 | 480 | 93.43 | 71.2 | 6.30 |

| | |
|---|---|
| F17 | Heterogenous; particles too large to measure |
| F18 | Heterogenous; particles too large to measure |

This analysis indicated that all samples processed with polyethylene wax (F01, F12) or carnauba wax (F02, F11, F20) exhibited unimodal distributions with volume-weighted mean particle size values (d_{4,3}) higher than that of the uncoated substrate, indicating the presence of an adhered coating layer with no residual coating material present. Conversely, all prototypes produced using ethylcellulose (F03, F13, F14) or the ethylcellulose system (F04) exhibited bimodal particle size distributions. The small-diameter secondary peaks seen in the ethylcellulose-containing products F13 and F14, combined with the high peak percentage of that mode, indicate the presence of residual coating material in the final product. The secondary modes exhibited by F03 and F04 were also likely due to unadhered coating particles; peak percentages for their secondary peaks are much smaller, owing to the lower coating level (20%) compared with that of F13 and F14 (40%).

Optical images of each carbohydrate-based extruded granule product produced with polyethylene wax and carnauba wax as the coating materials showed strong visual signs of particle coating, including smooth white spots of various thicknesses adhered to the carbohydrate-based extruded granule substrate particles. By comparison, it was clear from the images of the samples processed with ethylcellulose and the ethylcellulose system that these coating materials did not significantly deform during processing and largely remained as adhered but discrete powder particles. The carbohydrate-based extruded granule portion of each of the two samples processed with hydrogenated cottonseed oil appeared very similar in shape and texture to the unprocessed granule core, leaving little to no indication of particle coating. It was also observed from these two samples that large particles of raw hydrogenated cottonseed oil were still present in the final processed sample. However, the image of sample F18 also indicated the presence of particle agglomerates, likely formed via deposition of a softened or partially melted layer of fat on the substrate surface.

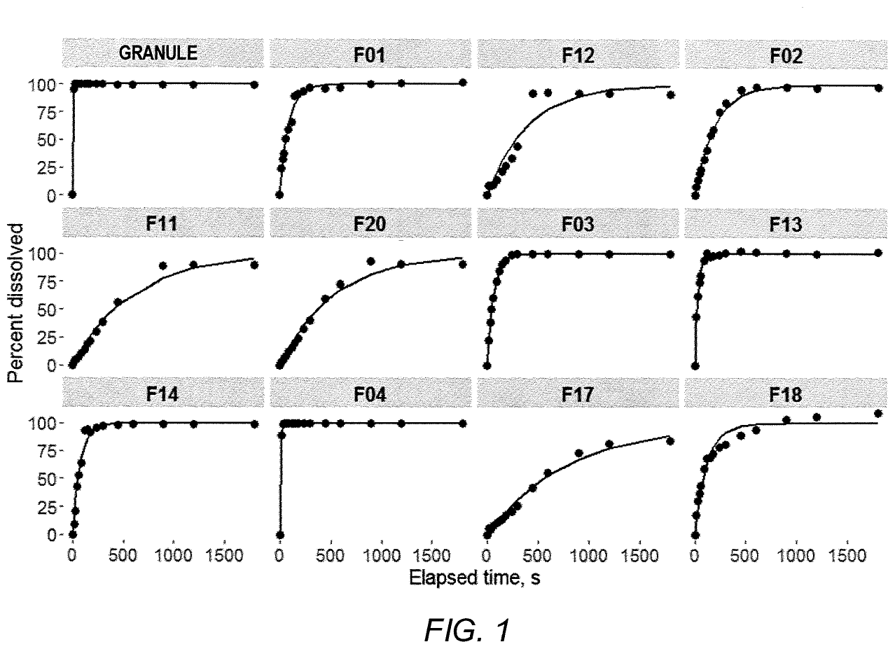

Dissolution testing results are depicted graphically in FIG. 1. These results confirmed many of the conclusions obtained from the particle size analysis and optical microscopy results. In particular, it is shown that the granule sample processed with the ethylcellulose system, F04, showed no signs of delayed release, with product release similar to that of the untreated granule substrate. The ethylcellulose sample, F13, also showed a very fast release, indicating absence of a continuous coating layer. However, the polyethylene wax and carnauba wax samples, F11 and F12, respectively, exhibit very extended release profiles, indicative of a cohesive coating layer with limited water diffusivity. In addition, sample F17, processed with hydrogenated cottonseed oil, exhibited the strongest delay in product release, showing less than 85% release after 30 minutes of dissolution time.

### EXAMPLE 3

### Coating of Carbohydrate-Based Spray-Dried Powder

Results of the particle size distributions of all samples manufactured using the spray-dried powder as a substrate are presented in Table 3.

TABLE 3

| | | Dominant Mode | | Secondary Mode | | Tertiary Mode | |
|---|---|---|---|---|---|---|---|
| | d_{4,3}, | Size, | Peak | Size, | Peak | Size, | Peak |
| Sample | μm | μm | % | μm | % | μm | % |
| Substrate | 59.8 | 54.6 | 96.25 | 6.86 | 3.75 | — | — |
| only | | | | | | | |
| F05 | 63.6 | 68.2 | 70.96 | 6.84 | 27.62 | 532 | 1.43 |
| F10 | 66.3 | 63.8 | 92.92 | 8.95 | 7.08 | — | — |
| F06 | 54.5 | 61.6 | 63.32 | 8.10 | 35.43 | 519 | 1.26 |
| F09 | 57.8 | 58.3 | 74.61 | 8.69 | 24.06 | 521 | 1.33 |
| F07 | 65.5 | 55.3 | 68.90 | 8.18 | 27.92 | 588 | 3.18 |
| F08 | 90.1 | 50.9 | 62.14 | 7.53 | 31.40 | 611 | 6.46 |
| F15 | 62.2 | 7.63 | 54.16 | 54.7 | 41.42 | 543 | 4.41 |
| F6 | 46.1 | 7.68 | 51.50 | 54.7 | 46.13 | 519 | 2.37 |

| | |
|---|---|
| F19 | Heterogenous; particles too large to measure |

In contrast to the results using carbohydrate-based extruded granule as, all spray-dried powder samples processed using the acoustic mixer, as well, as the core material itself, exhibited a particle size distribution with a minimum of two modes. In addition, eight of the nine measurable samples exhibited a trimodal distribution; sample F10 contained only two modes, while sample F19 was unable to be measured due to its very large final particle size. The presence of these additional modes, in conjunction with the high peak percentages of the secondary peak (generally in the particle size range of 6 μm to 9 μm), indicated the absence of particle coating on the spray-dried powder substrate.

With the exception of sample F19, optical microscopy images obtained for the other spray-dried powder processed samples appeared to show a loose association of the coating powder particles with the substrate particles. Specifically, individual particles of the coating material adhered to the surface of the substrate, but failed to significantly deform and form a cohesive, continuous coating, such as that seen in the granule samples processed with carnauba wax and polyethylene wax. Much of the substrate appeared to be exposed, lacking the protection afforded by a true coating. Notably, sample F19, processed with hydrogenated cottonseed oil as the coating material, had a unique structure and appearance. This sample visually appeared as large, opaque blue flakes, with very little evidence of residual spray-dried powder material. Due to the very small particle size of the spray-dried powder compared with the large particle size of the fat flakes, the processing of these two materials together resulted in spray-dried powder-coated fat flakes, i.e., an inverted form of the desired product. This result showed that, although the hydrogenated cottonseed oil is highly deformable, the spray-dried powder is also deformable and capable of forming a continuous layer via mechanical means alone.

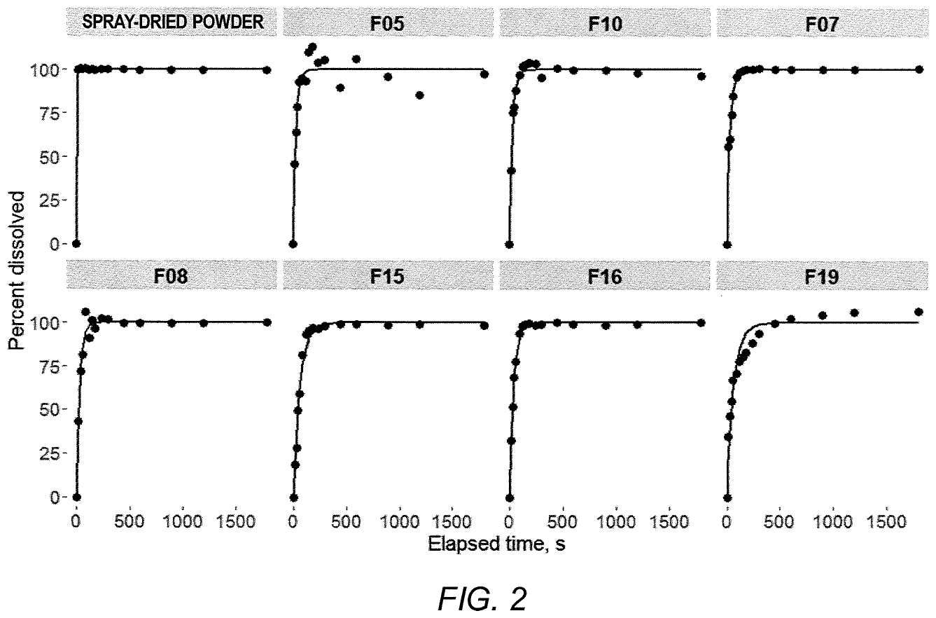

Dissolution results for spray-dried powder-based samples are presented graphically in FIG. 2. Dissolution results for the samples processed with carnauba wax (F06 and F09) were unable to be measured due to high solution turbidity, even after one week of solution settling. The dissolution curves for the remaining samples indicate that, while the spray-dried powder core still had the fastest release, no single sample exhibited strong delayed product release. Unexpectedly, sample F19 showed the slowest product release of all spray-dried powder-based samples, releasing 50% of the product in just under 45 seconds, compared to 1.8 seconds for the unprocessed spray-dried powder substrate. This result shows that the spray-dried powder mixed with the hydrogenated cottonseed oil in some manner, rather than forming a discrete layer on the surface of the fat particle.

### EXAMPLE 4

### Process Materials

A significant number of product properties play contributing roles in this dry acoustic coating process. The following analyzes the effects of formulation changes on the dissolution properties of the final product, and hence, the effectiveness of the acoustic mixing process for particle coating.

Coating Material Hardness. The primary factor contributing to the success of particle coating by way of acoustic mixing is the hardness of the coating material. It is known that the hydrogenated cottonseed oil is the softest and most easily deformable coating material used in this study, followed by carnauba wax, polyethylene wax and ethylcellulose, in order from softest to hardest. The differences in deformability of these materials and their ability to adhere to the substrate are reflected in the dissolution results shown in Table 4.

TABLE 4

| | Coating | Coating | | Delay | |
|---|---|---|---|---|---|
| Sample | Material | Level | Plasticizer | Rate | t₈₀, s |
| F13 | EC | 40% | — | 3.448 | 50.6 |
| F12 | PE | 40% | — | 5.999 | 648.5 |
| F11 | CW | 40% | — | 6.389 | 958.1 |
| F17 | CO | 40% | — | 6.694 | 1300 |
| F02 | CW | 20% | — | 5.277 | 315.0 |
| F11 | CW | 40% | — | 6.389 | 958.1 |
| F13 | EC | 40% | — | 3.448 | 50.6 |
| F14 | EC | 40% | 10% | 4.344 | 123.9 |

The product's delay rate and t₈₀ increased monotonically as a function of coating material hardness, indicating that mechanical deformability is critical to the success of particle coating under these conditions. As a result, coating of particles of this invention with soft polymers or gums, hard polymers such as hydroxypropyl cellulose and hypromellose, or crystalline materials such as salt and sucrose, would likely not be ideal.

Coating Level. An additional contributing factor to product properties is the coating level, or more directly, the fraction of coating material included in the sample. Table 5 shows a comparison between two granule samples processed with different levels of carnauba wax.

TABLE 5

| | Coating | Coating | | Delay | |
|---|---|---|---|---|---|
| Sample | Material | Level | Plasticizer | Rate | t₈₀, s |
| F02 | CW | 20% | — | 5.277 | 315.0 |
| F11 | CW | 40% | — | 6.389 | 958.1 |

The results of this analysis indicated that the assumed trend holds true for the acoustic mixing process. Although shown in the table with carnauba wax, this trend is expected to apply to other deformable coating materials as well, with the increase in delay rate directly related to the mechanical deformability or elastic modulus of the coating material.

Effect of Plasticizer. The incorporation of a suitable plasticizer is also shown to have an effect on the quality of the coating in the acoustic mixer (Table 6).

TABLE 6

| | Coating | Coating | | Delay | |
|---|---|---|---|---|---|
| Sample | Material | Level | Plasticizer | Rate | t₈₀, s |
| F13 | EC | 40% | — | 3.448 | 50.6 |
| F14 | EC | 40% | 10% | 4.344 | 123.9 |

Using ethylcellulose as the model coating material, an increase in delay rate and tso was shown in a sample processed with 10% triethyl citrate (TEC) over the sample processed in the absence of that plasticizer. These data likely indicate a mild softening of the coating during processing, enhancing the overall deformability of the coating and contributing to a greater surface coverage on the substrate particle. Sample F14 was prepared for acoustic processing simply by combining the granule substrate with ethylcellulose powder and stirring briefly, then adding triethyl citrate (a liquid at room temperature) and stirring again or alternatively spray drying the ethylcellulose and triethyl citrate together. Although the plasticized sample exhibited stronger delayed release properties, a better distribution of the triethyl citrate prior to processing in the acoustic mixer may produce even greater delayed release of the coated substrate.

It should be noted that the substrates used in these experiments were simple carbohydrate matrices and not true flavor delivery systems, as they both lacked any flavor oil. It is expected that the same results and trends that were found from testing with blank granule and spray-dried powder cores will be observed in these same systems containing encapsulated flavor oil, due to their having similar surface characteristics as the blank carbohydrate matrices. Additionally, processed granules and standard spray dry products would likely exhibit the same processing results as granule and spray-dried powder, respectively, due to their similar structure and character. However, those products containing higher levels of unencapsulated oils present on the surface of those particles would likely result in decreased coating efficiency due to potential surface lubricity effects.

### EXAMPLE 5

### Process Parameters

The combination of particle size analysis data, optical microscopy images and dissolution testing data elucidated not only the effect of the acoustic mixing operation on the products themselves, but also the means and mechanisms at play in the solventless mechanical coating process. The following describes how the process parameters contributed to the efficiency and success of the acoustic mixing technology for particle coating.

Processing Time. Table 7 shows two granule samples coated with carnauba wax for different amounts of time.

TABLE 7

| | Coating | Coating | Run | Delay | |
|---|---|---|---|---|---|
| Sample | Material | Level | time | Rate | t₈₀, s |
| F11 | CW | 40% | 4 h | 6.389 | 958.1 |
| F20 | CW | 40% | 2 h | 6.284 | 862.4 |

The dissolution results for these two samples exhibited the trend that processing the sample for a longer period of time in the acoustic mixer resulted in a greater delay in product release, indicating the presence of a more cohesive coating layer. However, it appeared that the increase in delay between a sample processed for two hours and one processed for four hours may not be as significant as initially expected. These results give some indication of the timescale of the coating process; it seemed that the majority of the coating process had already taken place after the first two hours of acoustic treatment. This may also be a function of the elastic modulus of the coating material, as one would expect that a more deformable material would form a cohesive coating on a shorter timescale than a harder material.

Effect of Impaction Media. Table 8 shows the effect of impaction media on the effectiveness of the acoustic coating process.

TABLE 8

| | Coating | Coating | Impaction | Delay | |
|---|---|---|---|---|---|
| Sample | Material | Level | Medium | Rate | t₈₀, s |
| F07 | EC | 30% | None | 3.341 | 45.5 |
| F08 | EC | 40% | 50% | 3.441 | 50.2 |

Samples F07 and F08 were spray-dried powder-based prototypes processed with ethylcellulose as the intended coating material. Sample F08 additionally contained 30 mesh glass beads as impaction media in order to provide additional collision force on the coated prototype and reduce the porosity of the coating layer. The dissolution results indicated that, while the sample processed with the impaction media did increase the delay rate of the processed prototype, that increase was very small; the presence of the glass beads improved the prototype's t80 value from 45.5 s to 50.2 s. While ethylcellulose was the hardest and least deformable of the coating materials used in the study, the inclusion of impaction media with a softer coating material is expected to have a significant impact. Additionally, a higher coating level may be required to fully coat the surface of the substrate particles; impaction media would serve to improve the cohesiveness of the coating. The presence of impaction media would also play a larger role in the compaction of processing time for certain coated prototypes. However, ideally, the size of the impaction media used is different than the size of the finished product, as separation of the impaction media from the product will likely be achieved through mechanical means (i.e., through sieving or filtration).

Vessel Fill Fraction. In Table 9, two hydrogenated cottonseed oil-containing granule prototypes with varying batch sizes were compared.

TABLE 9

| | Coating | Coating | Batch | Delay | |
|---|---|---|---|---|---|
| Sample | Material | Level | size | Rate | t₈₀, s |
| F17 | CO | 40% | 50 g | 6.694 | 1300 |
| F18 | CO | 40% | 100 g | 4.785 | 192.6 |

Sample F17 exhibited a much stronger delayed release than did sample F18, indicating that batch size is a major contributor to coating quality. These data indicated that the mean free path of particles in the sample vessel was a major governing factor dictating the success or failure of the coating process. Specifically, the larger batch size of F18 resulted in a higher vessel fill fraction, meaning that the mean free path of particles in the vessel was reduced relative to that in F17. Thus, the sample processed with a larger fill fraction (F18) experienced less forceful particle-particle and particle-wall collisions, likely resulting in a less cohesive coating layer.

In attempting to process samples with larger batch sizes, it was also seen that acoustic mixing intensity in the LabRAM was limited by the mass of the vessel and its contents. While 50 g prototypes could achieve the target mixing acceleration of 100 g at mixing intensity values below 100%, batches processed with 100 g sample in the vessel were only initially able to achieve accelerations of 75-85 g. Additionally, larger batch-size samples achieved higher final temperatures (approximately 45° C.) than smaller samples (approximately 35° C.). This correlation has been previously observed in the context of powder blending operations (Osorio & Muzzio (2015) Powder Technol. 278:46-56). Both of these phenomena are due to the tendency for products to absorb a certain fraction of the acoustic energy as heat, which fraction is directly related to the mass of the sample. However, all samples were able to achieve the desired 100 g intensity after some period of transition time.

What is claimed is:

1. A method for coating solid granules containing a carbohydrate, gum Arabic, or protein comprising
(a) combining solid granules containing a carbohydrate, gum Arabic, or protein with at least one solid coating material, and
(b) inducing vibrations in the combination of the solid carbohydrate granules and the solid coating material via acoustic energy thereby coating the solid granules.

2. The method of claim 1, wherein the carbohydrate of the solid granules comprises starch, maltodextrin, sugar, polyol, cellulose, cellulose derivatives, or a combination thereof.

3. The method of claim 1, wherein the at least one solid coating material comprises gum Arabic, cellulose, a cellulose derivative, a wax, a fat, polyol, sugar, protein or a combination thereof.

4. The method of claim 1, further comprising adding a plasticizer to the combination of the solid granules and the solid coating material.

5. The method of claim 4, wherein the plasticizer comprises an organic citrate salt, triglyceride, glycerol derivative, or a combination thereof.

6. The method of claim 1, further comprising adding impaction media to the combination of solid granules and the solid coating material.

7. The method of claim 6, wherein the impaction media comprise glass beads, metal balls, or a combination thereof.

8. The method of claim 1, wherein the solid granules or the solid coating material comprise an encapsulated flavor or encapsulated fragrance.

9. Coated solid granules produced by the method of claim

1.

10. The coated solid granules of claim 9, wherein the carbohydrate of solid granules comprises starch, maltodextrin, sugar, polyol, cellulose, cellulose derivatives, or a combination thereof.

11. The coated solid granules of claim 9, wherein the solid coating material comprises gum Arabic, cellulose, a cellulose derivative, carnauba wax, polyethylene wax, polyol, sugar, protein or a combination thereof.

12. The coated solid granules of claim 9, further comprising a plasticizer.

13. The coated solid granules of claim 12, wherein the plasticizer comprises an organic citrate salt, triglyceride, glycerol derivative, or a combination thereof.

14. The coated solid granules of claim 9, further comprising an encapsulated flavor or encapsulated fragrance.

15. Coated solid granules produced by the method of claim 8.
